(19) United States
(12) Patent Application Publication
(10) Pub. No.: US 2009/0163856 A1
(43) Pub. Date: Jun. 25, 2009

(54) Treatment indications informed by a prior implant information

(75) Inventors: Bran Ferren, Beverly Hills, CA (US); Roderick A. Hyde, Redmond, WA (US); Muriel Y. Ishikawa, Livermore, CA (US); Eric C. Leuthardt, St. Louis, MO (US); Dennis J. Rivet, Portsmouth, VA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/004,453

(22) Filed: Dec. 19, 2007

(51) Int. Cl. A61M 31/00

(52) U.S. Cl. 604/66

(57) ABSTRACT

Systems and methods are described for implementing or deploying therapeutic administration systems for obtaining a priori implant information and signaling a decision whether to initiate implant-site-targeting treatment partly based on the a priori implant information and partly based on one or more other clot-indicative determinants; or obtaining a flow-change-indicative measurement and signaling a decision whether to administer one or more clot-reducing agents at least partly based on the flow-change-indicative measurement; or obtaining one or more indications of a lytic material in a vicinity of one or more body lumens and accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens; or one or more capture components configured to accelerate a decrease in a local concentration of one or more therapeutic structures along a downstream portion of a vasculature and one or more dispensation components configured to release the one or more therapeutic structures into an upstream portion of the vasculature.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)).

### SUMMARY

In one aspect, a method includes but is not limited to obtaining a priori implant information and signaling a decision whether to initiate implant-site-targeting treatment partly based on the a priori implant information and partly based on one or more other clot-indicative determinants. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining a priori implant information and circuitry for signaling a decision whether to initiate implant-site-targeting treatment partly based on the a priori implant information and partly based on one or more other clot-indicative determinants. In addition to the foregoing, other system aspects are described in the claims, drawings, and text forming a part of the present disclosure.

In one aspect, a method includes but is not limited to obtaining a flow-change-indicative measurement and signaling a decision whether to administer one or more clot-reducing agents at least partly based on the flow-change-indicative measurement. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining a flow-change-indicative measurement and circuitry for signaling a decision whether to administer one or more clot-reducing agents at least partly based on the flow-change-indicative measurement. In addition to the foregoing, other system aspects are described in the claims, drawings, and text forming a part of the present disclosure.

In one aspect, a method includes but is not limited to obtaining one or more indications of a lytic material in a vicinity of one or more body lumens and accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining one or more indications of a lytic material in a vicinity of one or more body lumens and circuitry for accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens. In addition to the foregoing, other system aspects are described in the claims, drawings, and text forming a part of the present disclosure.

In one aspect, a system includes but is not limited to one or more capture components configured to accelerate a decrease in a local concentration of one or more therapeutic structures along a downstream portion of a vasculature and one or more dispensation components configured to release the one or more therapeutic structures into an upstream portion of the vasculature. In addition to the foregoing, other system aspects are described in the claims, drawings, and text forming a part of the present disclosure.

In one or more various aspects, related systems include but are not limited to circuitry and/or programming for effecting herein-referenced method aspects; the circuitry and/or programming can be virtually any combination of hardware, software, and/or firmware configured to effect the herein-referenced method aspects depending upon the design choices of the system designer. In addition to the foregoing, various other method and/or system aspects are set forth and described in the teachings such as text (e.g., claims and/or detailed description) and/or drawings of the present disclosure.

The foregoing is a summary and thus contains, by necessity, simplifications, generalizations and omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIGS. 1-2 depict exemplary environments in which one or more technologies may be implemented.

FIG. 3 depicts a high-level logic flow of an operational process.

FIG. 4 depicts an exemplary environment in which one or more technologies may be implemented.

FIG. 5 depicts a high-level logic flow of an operational process.

FIG. 6 depicts an exemplary environment in which one or more technologies may be implemented.

FIG. 7 depicts a high-level logic flow of an operational process.

FIGS. 8-12 depict respective contexts in which one or more medical or veterinary technologies as described herein may be implemented.

FIGS. 13-14 depict variants of the flow of FIG. 3.

FIG. 15 depicts variants of the flow of FIG. 5.

FIGS. 16-17 depict variants of the flow of FIG. 7.

### DETAILED DESCRIPTION

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will tropically employ optically-oriented hardware, software, and or firmware.

In the following detailed description, reference is made to the accompanying drawings, which form a part hereof. The use of the same symbols in different drawings tropically indicates similar or identical items. The illustrative embodiments described in the detailed description, drawings, and claims are not meant to be limiting. Other embodiments may be utilized, and other changes may be made, without departing from the spirit or scope of the subject matter presented here.

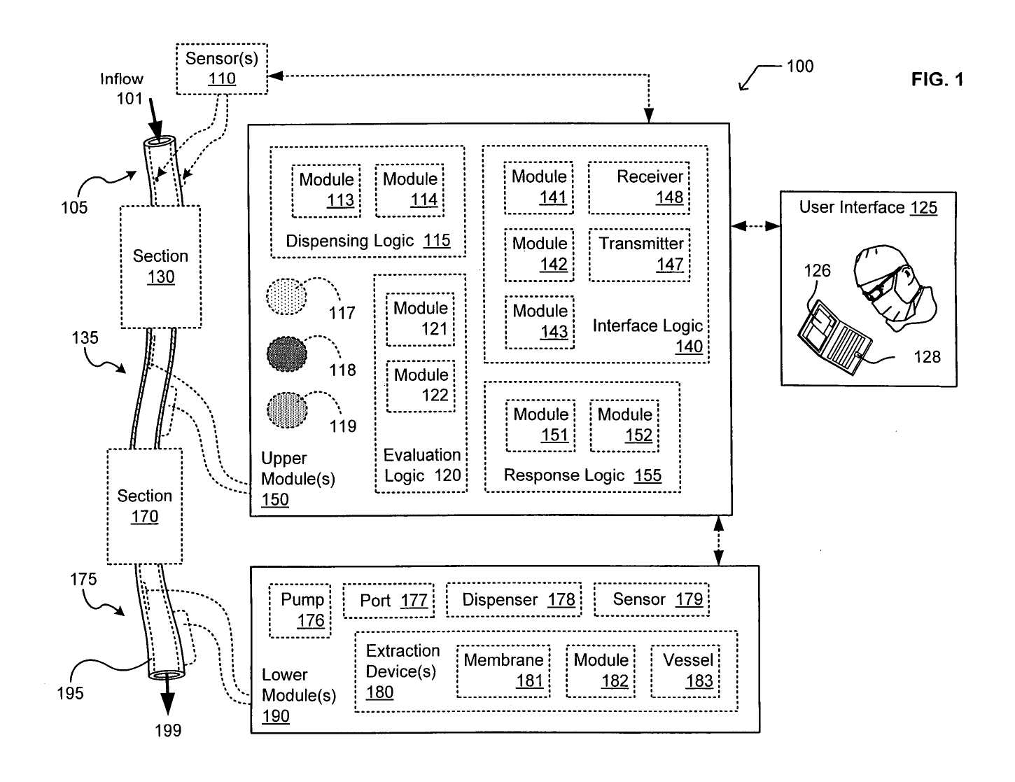

With reference now to FIG. 1, shown is an example of a system that may serve as a context for introducing one or more processes and/or devices described herein. As shown system 100 may affect or otherwise relate to vicinity 105, section 130, vicinity 135, section 170, and vicinity 175 of a vascular lumen 195 through which one or more blood components may flow. One or more inflows 101 of blood enter respective portions of lumen 195 as shown, pass through sections 130, 170 and exit as one or more outflows 199. In respective variants, arteries, veins, or smaller vessels of lumen 195 may traverse proximities 105, 135, 175 as shown. Sections 130, 170 may likewise comprise one or more capillary beds as well as implants or other entities with which lumen 195 interacts.

In some variants, one or more upper modules 150 in vicinity 135 may (optionally) send data to and/or receive data from one or more instances of intravascular or other sensors 110 in vicinity 105. Upper module 150 may likewise comprise one or more instances of modules 113, 114 of dispensing logic 115; dispensers 117, 118, 119; modules of evaluation logic 120; transmitters 147, receivers 148, or other modules 141, 142, 143 of interface logic 140; or modules 151, 152 of response logic 155. Interface logic may (optionally) handle data to output device 126 and/or from input device 128 as well interacting with one or more lower modules 190. Lower module 190 may include one or more instances of microfluidic or other pumps 176, ports 177, dispensers 178, sensors 179, or semi-permeable membranes 181 or other such modules 182 or vessels 183 of extraction devices 180.

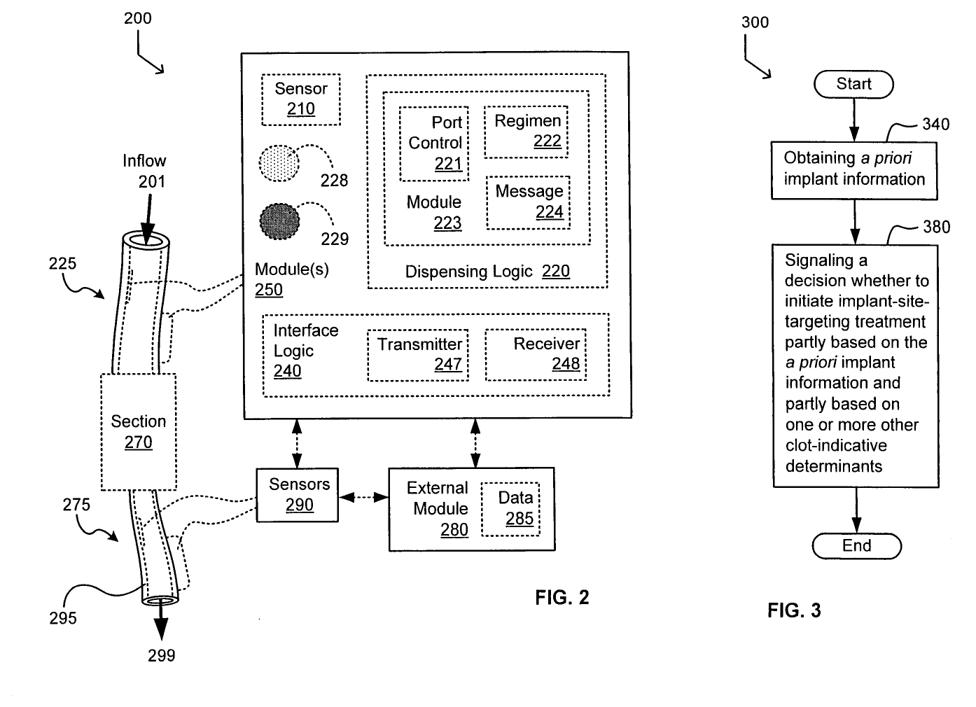

With reference now to FIG. 2, shown is an example of a system that may serve as a context for introducing one or more processes and/or devices described herein. As shown system 200 may affect or otherwise relate to vicinity 225, section 270, and vicinity 275 of a vascular lumen 295 through which one or more blood components may flow. One or more inflows 201 of blood enter respective portions of lumen 295 as shown, pass through section 270, and exit as one or more outflows 299. In respective variants, arteries, veins, or smaller vessels of lumen 295 may traverse proximities 225, 275 as shown. Section 270 may likewise comprise one or more capillary beds as well as vital organs and other tissues served by lumen 295.

In some variants, one or more intravascular or other modules 250 in vicinity 225 may (optionally) include one or more instances of sensors 210; modules 223 or other dispensing logic 220; dispensers 228, 229; or transmitters 247, receivers 248, or other interface logic 240. (Some such modules 250 may be operable for penetrating a vascular structure with ultrasonic or other energy, for example, or may comprise an implanted cannula or other transvascular structure.) Module 223 may, as shown, comprise one or more instances of port controls 221, regimens 222 or other programmatic dispensing information (optionally embodied in software or other instruction sequences, for example), or requests or other messages 224.

Alternatively or additionally, system 200 may comprise one or more intravascular or other sensors 290 that may (optionally) be configured to communicate (in one or both directions) with module 250, such as by a signal-bearing conduit or radio-frequency signal. (Some such sensors 290 may be operable for monitoring one or more physical phenomena within vascular structures, for example, from within or in a vicinity of the structures.) Systems 200 may likewise be configured to include or otherwise interact with one or more instances of external modules 280 operable, for example, for obtaining and providing data 285 as described herein. In some variants, for example, the one or more sensors 290 are only operable for communicating sensed analog or digital values to module 250. In others, one or more of the sensor(s) 290 are able to receive updates or other information from one or more external modules 280 or other transmitters 247 as described herein.

With reference now to FIG. 3, shown is a flow 300 comprising operation 340—obtaining a priori implant information (e.g. receiver 248 receiving user-provided or other data 285 describing one or more sensors 290 or other implants downstream from one or more modules 250 in a vicinity 275 of lumen 295). This can occur, for example, in a context in which module 250 comprises a cannula or other implantable structure positioned upstream from an outflow 299 local to the implant(s) to which the a priori information pertains. Alternatively or additionally, receiver 248 may obtain sensor data or other determinants relating to such implants, as described herein.

Flow 300 further comprises operation 380—signaling a decision whether to initiate implant-site-targeting treatment partly based on the a priori implant information and partly based on one or more other clot-indicative determinants (e.g. interface logic 240 invoking one or more modules 223 of dispensing logic 220 operable for activating one or more dispensers 228 containing one or more thrombolytic agents or other locally-administered therapeutic materials selectively when apparently needed in a vicinity 275 of lumen 295). This can occur, for example, in a context in which the a priori implant information indicates a drug-eluting stent or other potential thrombogenic implant at outflow 299.

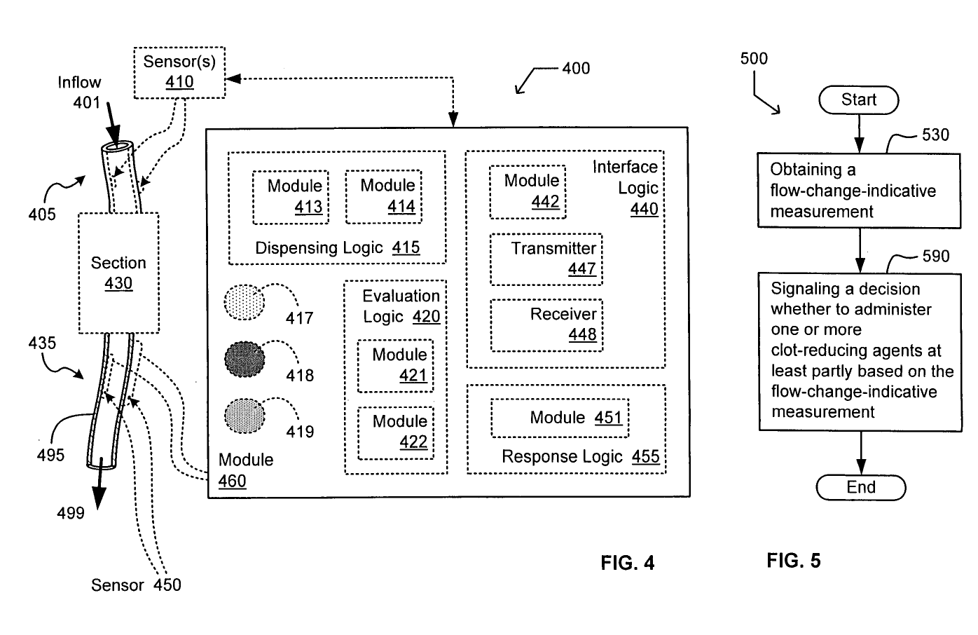

With reference now to FIG. 4, shown is an example of a system that may serve as a context for introducing one or more processes and/or devices described herein. As shown system 400 may affect or otherwise relate to vicinity 405, section 430, and vicinity 435 of a subject's lumen 495 through which one or more blood components may flow. One or more inflows 401 of blood enter respective portions of lumen 495 as shown, pass through section 430, and exit as one or more outflows 499. In respective variants, arteries, veins, or smaller vessels of lumen 495 may traverse proximities 405, 435 as shown. Section 430 may likewise comprise one or more capillary beds as well as vital organs and other tissues served by lumen 495.

In some variants, module 460 may (optionally) include one or more instances of modules 413, 414 of dispensing logic 415; dispensers 417, 418, 419; modules 421, 422 of evaluation logic 420; interface logic 440; modules 451 or other response logic 455; or intravascular or other sensors 450. (Some such sensors 450 may be operable for monitoring radiant or other physical phenomena within a lumen 495, for example, from within or in a detection vicinity 405 of lumen 495.) Interface logic 440 may, as shown, comprise one or more instances of transmitters 447, receivers 448, or other modules 442 operable for communicating (in one or both directions) with one or more sensors 410 in (upstream) vicinity 405 of lumen 495.

With reference now to FIG. 5, shown is a flow 500 comprising operation 530—obtaining a flow-change-indicative measurement (e.g. one or more modules 421 of evaluation logic 420 detecting abnormally frequent blood pressure fluctuations for days consecutively). This can occur, for example, in a context in which a blood pressure fluctuation distribution for a specific pressure sensor is empirically determined and in which module 421 implements a threshold or other baseline derived by a reasonable statistical model. In some variants, for example, an appropriate normality threshold may be selected so that a frequency of occurrence or other measurable variable will be expected only to exceed the threshold once per decade (or similar duration within 1-2 orders of magnitude. Alternatively or additionally, a triggering condition may be selected in relation to one or more of optical, force, auditory, or other measurable criteria or to a combination of such criteria. Numerous reasonable triggering conditions will readily be apparent to those skilled in the art without undue experimentation, many of which are a mere matter of design choice in light of teachings herein.

Flow 500 further comprises operation 590—signaling a decision whether to administer one or more clot-reducing agents at least partly based on the flow-change-indicative measurement (e.g. one or more modules 413, 414 of dispensing logic 415 causing one or more dispensers 417, 418 to administer an antiplatelet-drug-containing or other therapeutic agent in response to the one or more modules 421, 422 of evaluation logic 420). This can occur, for example, in a context in which module 414 specifically selects such a therapeutic material by selecting the dispenser 418 containing the material in lieu of another dispenser. Alternatively or additionally, one or more modules 442 may be configured to signal the decision in some other way, such as by a speaker or other transmitter 447 conveying medication instructions to the (implanted) subject, or otherwise by sending such a message to a party who is able to implement the decision.

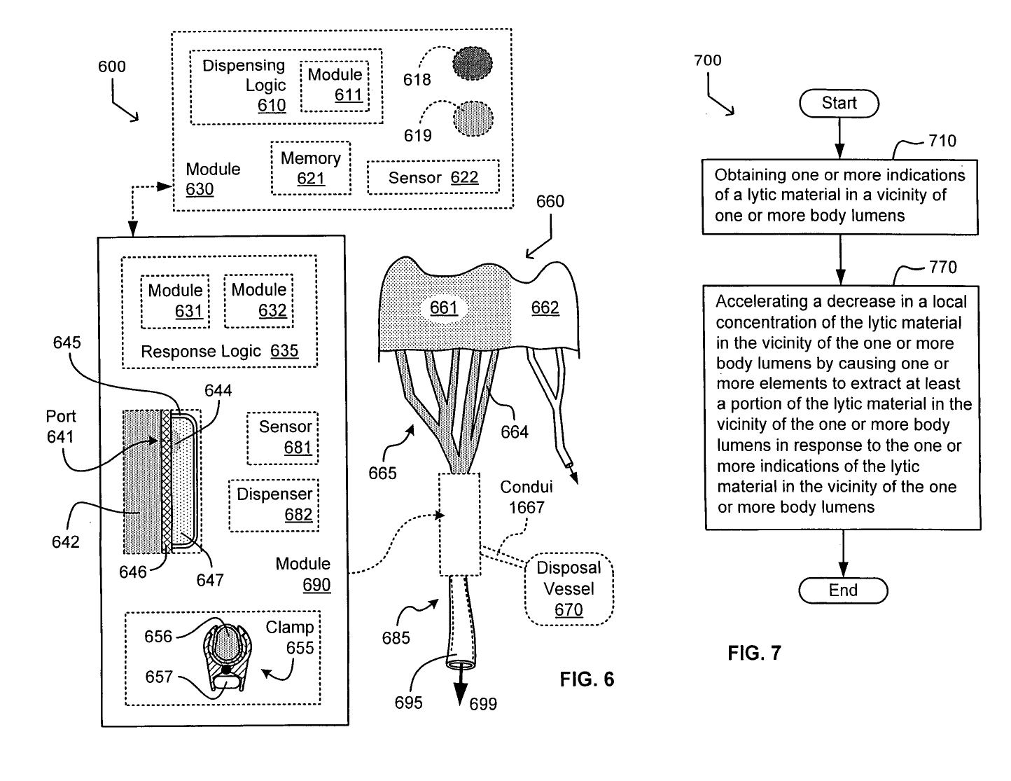

With reference now to FIG. 6, shown is an example of a system 600 that may serve as a context for introducing one or more processes and/or devices described herein, comprising one or more instances of module 630 operable for interacting with module 690. As shown, module 630 may include one or more modules 611 of dispensing logic 610 operable for controlling statin dispenser 618 or (other) therapeutic dispenser 619; memory 621 operable for handling software-implemented or other regimens; or one or more sensors 622 as described herein. Also shown is a kidney or other organ 660 having one or more (therapeutic-agent-) suffused portions 661 and one or more other portions 662, at least one of the suffused portions 661 comprising a vicinity 665 of (converging venules 664 of) lumen 695

Next downstream as shown, module 690 comprises one or more modules 631, 632 of response logic 635; (transvascular or other) extraction modules 645; sensors 681; dispensers 682; or clamps 655. As shown, extraction module 645 comprises one or more ports 641 to be formed through vessel wall 646, operable for extracting a portion 644 of lytic-material-infused blood 642, for example, into one or more absorbent elements 647 and/or to other disposal vessels at a lower-than-ambient pressure. As shown, one or more clamps 655 are configured to limit outflow 699 from module 690 by expanding one or more actuators 657, thereby levering lumen 695 to occlude it temporarily as shown. Alternatively or additionally, vicinity 685 of lumen 695 may include one or more conduits 667 operable for selectively removing a portion of outflow 699 by redirecting it to one or more artificial disposal vessels 670 as shown.

With reference now to FIG. 7, shown is a flow 700 comprising operation 710—obtaining one or more indications of a lytic material in a vicinity of one or more body lumens (e.g. module 631 of response logic 635 responding to a signal from one or more sensors 622, 681 or some other indication that an anticoagulant or other lytic material will apparently be present in a vicinity 665 of lumen 695). This can occur, for example, in a context in which response logic 635 receives a notification that one or more lytic-material-containing dispensers 619 have been activated. Alternatively or additionally, such indications can result from one or more sensors 681 detecting one or more natural chemical markers resulting from injury, for example. Alternatively or additionally, such indications can result from dispenser 682 administering a lytic compound by backflow into organ portion 661—injecting the compound at a somewhat higher pressure than that of blood in venules 664.

Flow 700 further comprises operation 770—accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens (e.g. port 641 or conduit 667 opening shortly after a dispensation of fibrinolytic material in upstream vicinity). This can occur, for example, in embodiments in which such ports or conduits are configured to allow higher-than-nominal concentrations of the lytic material to drain out of the vascular system, optionally by a timely exposure to an absorbent element 647 or other disposal vessel 670. Alternatively or additionally, such extraction may (optionally) be performed actively, such as by microfluidic or other pumps as described herein.

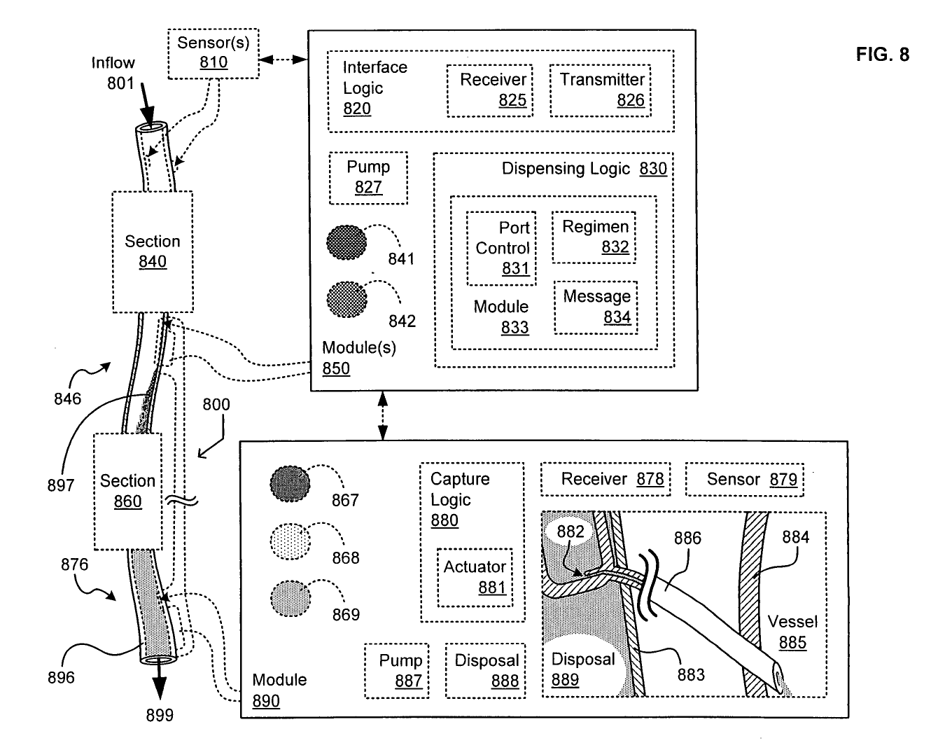

With reference now to FIG. 8, shown is an example of a system that may serve as a context for introducing one or more processes and/or devices described herein. As shown system 800 may affect or otherwise relate to one or more sections 840 or other “upstream” portions 846 of a human or other living subject's vasculature 896 (receiving inflow 801) and also to one or more “downstream” portions 876 of such vasculatures 896 (bearing outflow 899). One or more sections 840, 860 as shown may comprise one or more of capillary beds, tissues served by vasculature 896, or larger blood vessels as described above.

In some variants, one or more intravascular or other modules 850 may (optionally) include one or more instances of receivers 825, transmitters 826, or other interface logic 820 such as for communicating (in one or both directions) with one or more sensors 810 operable for monitoring upstream portion 846. Module 850 may likewise include one or more instances pumps 827 or other hardware controlled by dispensing logic 830 for selectively releasing one or more (biological, radiotherapy, or other) agents 841 or other therapeutic structures 842 into upstream portion 846. Such module(s) 850 may also be configured, in some contexts, by including one or more software or other modules 833 of dispensing logic 830 comprising one or more instances of port controls 831, (dispensing or other therapeutic) regimens 832, or messages 834 as described below.

As shown, system 800 may comprise one or more modules 850 upstream operable for communicating (in one or both directions) with one or more intravascular or other modules 890 downstream, optionally in an integral and/or implanted structure as shown. Alternatively or additionally, module 890 may include one or more instances of capture agents 867, 868 or other therapeutic agents 869; receivers 878; sensors 879; capture logic 880 operable for controlling one or more actuators 881, such as for optically or otherwise controlling the capture agent(s); pumps 887; or disposals 888, 889. As shown, for example, disposal 889 may include one or more ports 882 operable for accelerating a decrease in a local concentration of the agent(s) 841 or other therapeutic structure(s) 842 along portion 876 (downstream from dispensation 897, as shown) by allowing the structure(s) to pass into one or more conduits 886 traversing one or more vessel walls 883, 884. One or more vessels 885 configured to receive the structure(s) may include, in some embodiments, an esophagus or other natural vessels, implanted vessels, or ex situ vessels. Concerning the opening of port 641 or other timing of capture logic 880 or similar responsive circuitry described herein, a delay time between a capture site and an upstream site can be readily estimated with fair vasculature. A human blood cell typically travels about ⅓ of a millimeter per second in capillaries, for example. In some contexts, an accurate model may best be developed by measuring a specific interpositional delay empirically using, for example, a fluorescent material or other detectable measurement technique. Such a delay can readily be implemented in a digital or other timing feature of modules as described herein, for example, initiating a later operation at a programmed interval following a triggering event as described herein. In situations where a more reliable model is needed, a pulse-dependent, local-pressure-dependent, or other adaptive model may be appropriate, and well within the capabilities of skilled practitioners without undue experimentation in light of teachings herein.

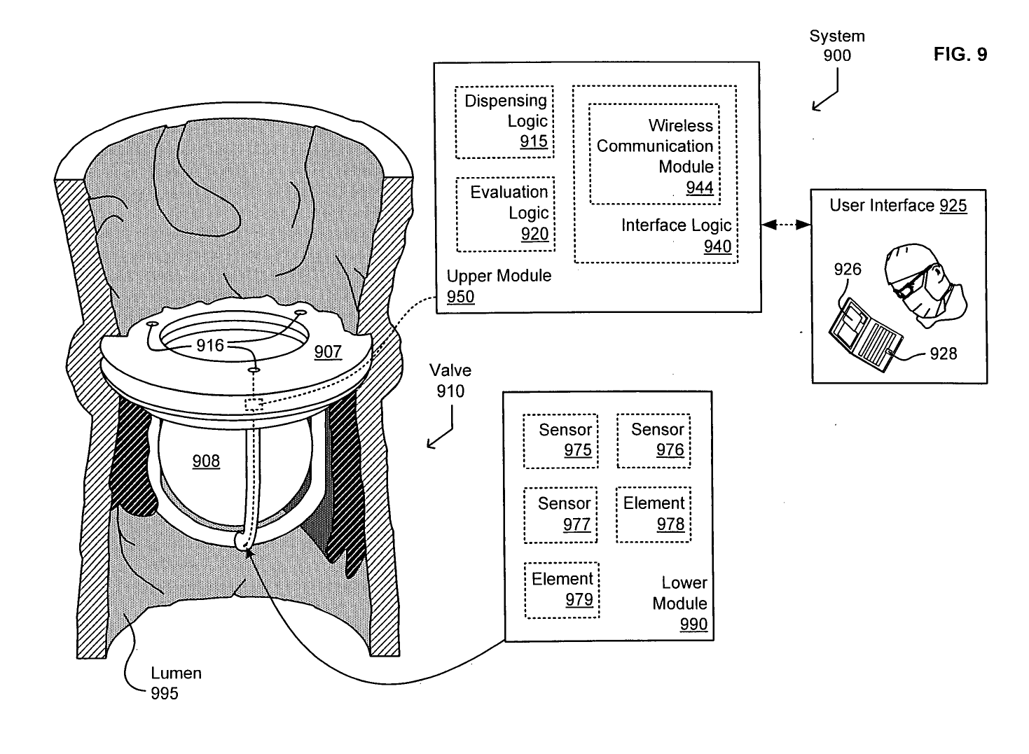

With reference now to FIG. 9, shown is an example of a system that may serve as a context for introducing one or more processes and/or devices described herein. As shown system 900 may comprise a lumen 995 comprising a heart valve 910 including an annular base 907 containing one or more dispensers 916, a ball 908, and one or more upper modules 950 and lower modules 990 operatively coupled as shown. Upper module 950 may comprise one or more instances of dispensation logic 915, evaluation logic 920, or wireless communication modules 944 or other interface logic 940 operable for communication with one or more user interfaces 925; for transmitting data to one or more output devices 926 or receiving data from one or more input devices 928 thereof as shown. Lower module 990 may comprise an optical sensor 975, an auditory sensor 976, or other sensors 977; or pressure or force sensors or other a flow-force-responsive elements 978 or other elements 979 as described herein.

An embodiment provides a system 900 comprising dispensing logic 915 or interface logic 940 operable for signaling a decision whether to initiate implant-site-targeting treatment and one or more dispensers 916 responsive to the decision. Each dispenser 916 may (optionally) include a thrombolytic agent and/or other therapeutic materials as described herein, suitable for targeting a vicinity of valve 910. The above-described “signaling” circuitry may comprise one or more of optical sensors 975, auditory sensors, flow-force-responsive elements 978, or other components suitable for providing thrombus-indicative measurements or other data suitable for informing the decision in light of teachings herein.

An embodiment provides a system 900 comprising interface logic 940 operable for signaling a decision (a) whether to initiate implant-site-targeting treatment or (b) whether to administer one or more clot-reducing agents. Alternatively or additionally, system 900 comprising may similarly provide dispensing logic using such signaling, for example, for guiding one or more dispensers 916 accordingly. Each dispenser 916 may (optionally) contain a thrombolytic agent and/or other therapeutic materials as described herein, suitable for targeting a vicinity of valve 910. The above-described “signaling” circuitry may comprise one or more of optical sensors 975, auditory sensors, flow-force-responsive elements 978, or other components suitable for providing thrombus-indicative measurements or other data suitable for informing the decision in light of teachings herein.

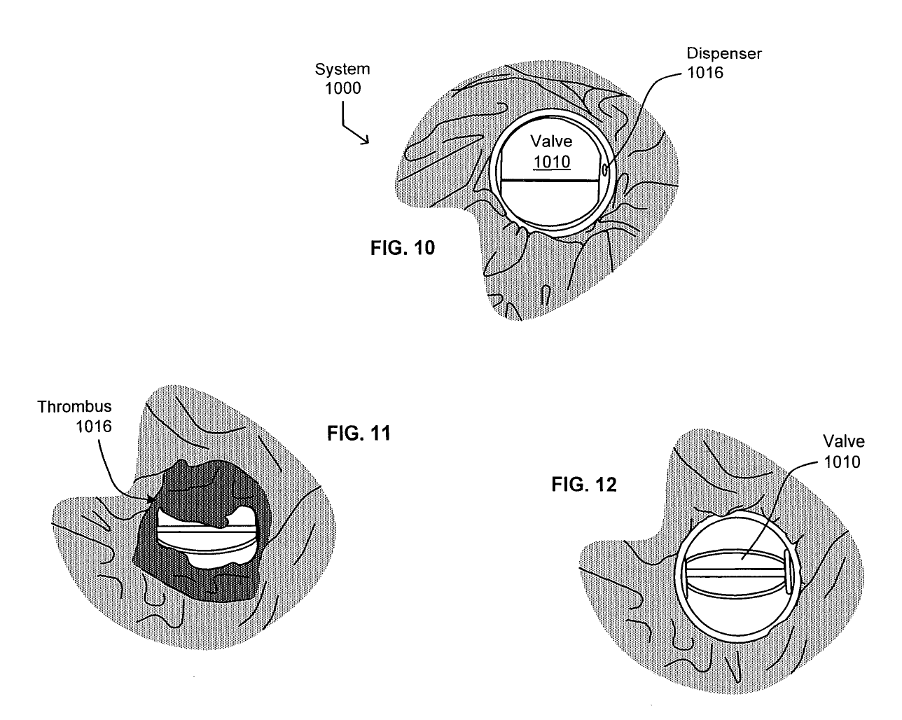

With reference now to FIG. 10, shown is an example of a system that may serve as a context for introducing one or more processes and/or devices described herein. As shown system 1000 comprises (a top view of) a valve 1010 having a dispenser 1016 in an upper portion thereof. Any of the embodiments described herein with reference to FIG. 1 may effectively implement valve 1010 as a combination of upper module 150 and lower module 190 within lumen 195. Any of the embodiments described herein with reference to FIG. 2 may effectively implement valve 1010 as module 250 within lumen 295. Any of the embodiments described herein with reference to FIG. 4 may effectively implement valve 1010 as module 460 within lumen 495. Any of the embodiments described herein with reference to FIG. 6 may effectively implement valve 1010 as module 690 within lumen 695. Any of the embodiments described herein with reference to FIG. 6 or 8 may likewise implement valve 1010 as module 690 or system 800 within lumen 695 or vasculature 896.

With reference now to FIG. 11, shown is (a bottom view of) a variant of valve 1010 in which a dangerous, partially occlusive thrombus 1016 has formed. An embodiment provides one or more sensors 179 in a lower module 190 suitable for detecting thrombus 1016 and able to respond programmatically as described herein.

With reference now to FIG. 12, shown is (a bottom view of) a variant of valve 1010 in which thrombus 1016 has been prevented or removed as described herein. Valve 1010 is according operable for opening and closing effectively in this configuration, unlike that of FIG. 11.

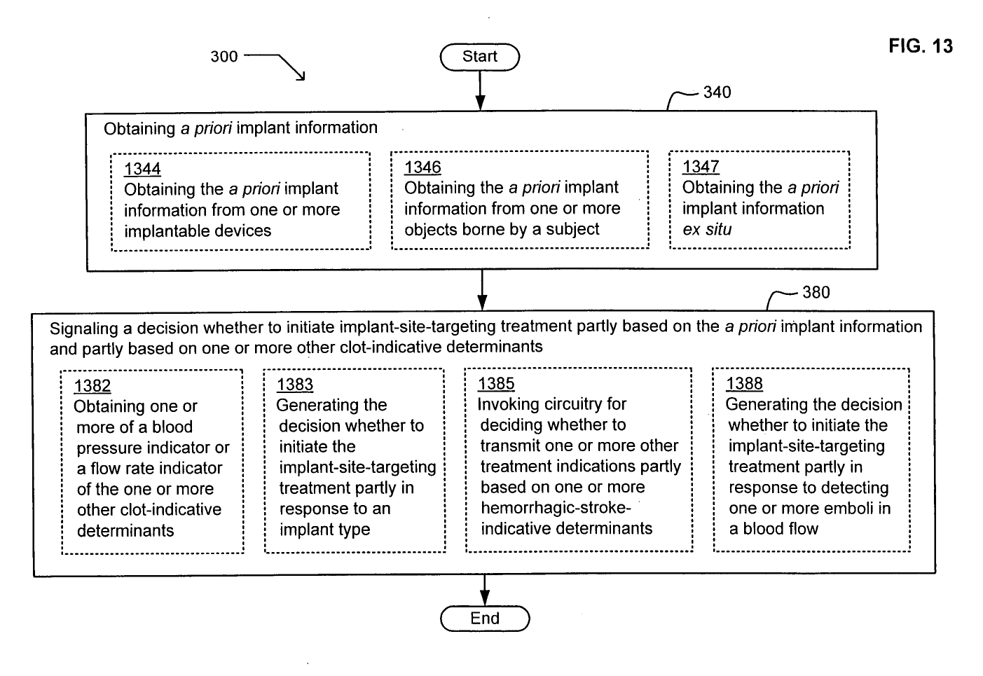

With reference now to FIG. 13, there are shown several variants of the flow 300 of FIG. 3. Operation 340—obtaining a priori implant information—may (optionally) include one or more of the following operations: 1344, 1346, or 1347. In some embodiments, variants of operation 340 may (optionally) be performed by one or more instances of dispensing logic 115, 220, receivers 148, 248, or the like as exemplified herein. Operation 380—signaling a decision whether to initiate implant-site-targeting treatment partly based on the a priori implant information and partly based on one or more other clot-indicative determinants—may include one or more of the following operations: 1382, 1383, 1385, or 1388. In some embodiments, variants of operation 380 may be performed by one or more instances of dispensers 119, 229, transmitters 147, 247, or the like as described herein.

Operation 1344 describes obtaining the a priori implant information from one or more implantable devices (e.g. external module 280 receiving specifications or other data 285 about module 250 from a wireless or other transmitter 247 thereof). This can occur, for example, in a context in which external module 280 notifies locally-available caregivers of the existence of module 250 and/or of dispensations or dosages from it. Such information may be used to expedite care or avoid redundant dispensations, for example. Operation 1346 describes obtaining the a priori implant information from one or more objects borne by a subject. Operation 1347 describes obtaining the a priori implant information ex situ.

Operation 1382 describes obtaining one or more of a blood pressure indicator or a flow rate indicator of the one or more other clot-indicative determinants. Operation 1383 describes generating the decision whether to initiate the implant-site-targeting treatment partly in response to an implant type. Operation 1385 describes invoking circuitry for deciding whether to transmit one or more other treatment indications partly based on one or more hemorrhagic-stroke-indicative determinants. Operation 1388 describes generating the decision whether to initiate the implant-site-targeting treatment partly in response to detecting one or more emboli in a blood flow. Any of these operations may be omitted or performed before or during one or more instances or variants of operation 340 as described above, for example.

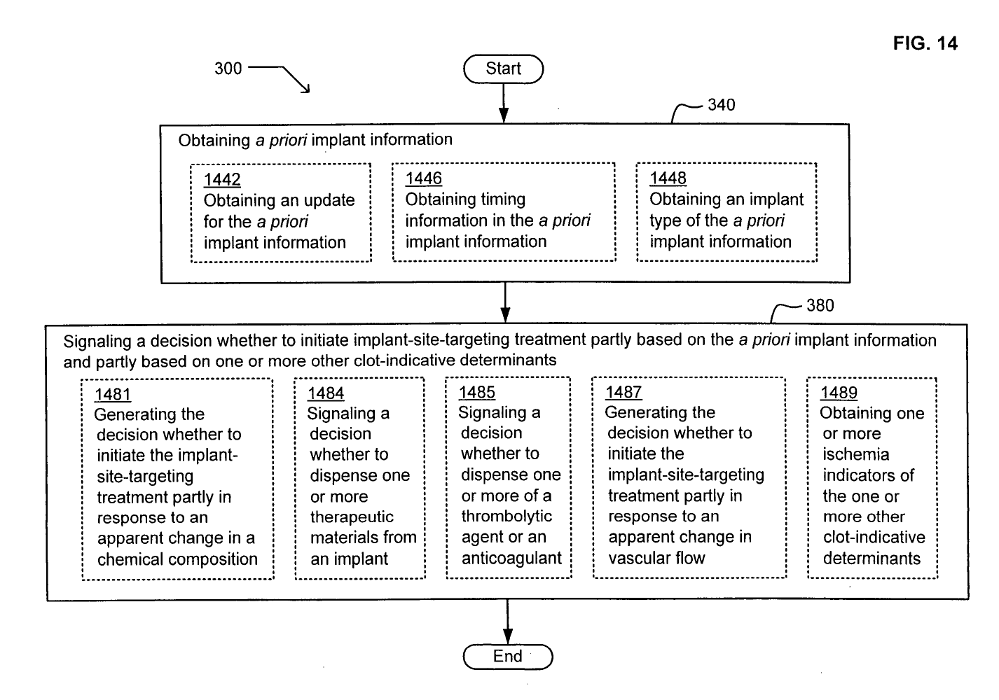

With reference now to FIG. 14, there are shown several variants of the flow 300 of FIG. 3 or 13. Operation 340—obtaining a priori implant information—may include one or more of the following operations: 1442, 1446, or 1448. In some embodiments, variants of operation 340 may be performed by one or more instances of dispensing logic 115, 220, receivers 148, 248, or the like as exemplified herein. Operation 380—signaling a decision whether to initiate implant-site-targeting treatment partly based on the a priori implant information and partly based on one or more other clot-indicative determinants—may include one or more of the following operations: 1481, 1484, 1485, 1487, or 1489. In some embodiments, variants of operation 380 may be performed by one or more instances of dispensers 119, 229, transmitters 147, 247, or the like as described herein.

Operation 1481 describes generating the decision whether to initiate the implant-site-targeting treatment partly in response to an apparent change in a chemical composition e.g. module 223 of dispensing logic 225 causing transmitter 247 to transmit a message 224 indicating one or more treatment materials 228, 229 and/or a dispensation site 226 local to section 270 as a programmatic response to an apparently severe hypoxic condition or other circumstance detected via one or more sensors 210, 290 operable for detecting chemical concentrations). This can occur, for example, in a context in which a caregiver can validate and/or administer the dispensation of such a treatment material via an intravenous catheter. Alternatively or additionally, the decision to administer an already-implanted material may be performed according to a programmatic crisis-response regimen 222 specified in advance by a caregiver in response to an abnormally high platelet concentration detected locally, for example, by sensor 210.

Operation 1484 describes signaling a decision whether to dispense one or more therapeutic materials from an implant. Operation 1485 describes signaling a decision whether to dispense one or more of a thrombolytic agent or an anticoagulant. Operation 1487 describes generating the decision whether to initiate the implant-site-targeting treatment partly in response to an apparent change in vascular flow. Operation 1489 obtaining one or more ischemia indicators of the one or more other clot-indicative determinants.

Operation 1442 describes obtaining an update for the a priori implant information. Operation 1446 describes obtaining timing information in the a priori implant information. Operation 1448 describes obtaining an implant type of the a priori implant information. Any of these operations may be omitted or performed before, after, or interleaved with one or more instances or variants of operation 380 as described above, for example.

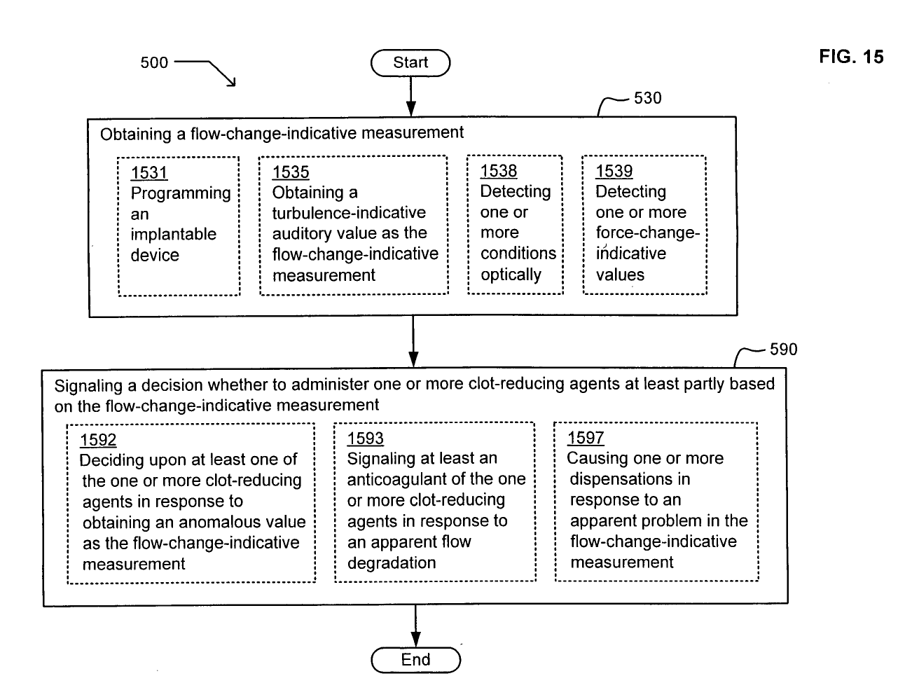

With reference now to FIG. 15, there are shown several variants of the flow 500 of FIG. 5. Operation 530—obtaining a flow-change-indicative measurement—may (optionally) include one or more of the following operations: 1531, 1535, 1538, or 1539. In some embodiments, variants of operation 530 may (optionally) be performed by one or more instances of sensors 179, 450, evaluation logic 120, 420, or the like as exemplified herein. Operation 590—signaling a decision whether to administer one or more clot-reducing agents at least partly based on the flow-change-indicative measurement—may include one or more of the following operations: 1592, 1593, or 1597. In some embodiments, variants of operation 590 may be performed by one or more instances of output devices 126, dispensing logic 115, 415, or the like as described herein.

As FIG. 15 indicates, (optional) operation 1531 describes programming an implantable device. Operation 1535 describes obtaining a turbulence-indicative auditory value as the flow-change-indicative measurement. Operation 1538 describes detecting one or more conditions optically. Operation 1539 describes detecting one or more force-change-indicative values. Operation 1592 describes deciding upon at least one of the one or more clot-reducing agents in response to obtaining an anomalous value as the flow-change-indicative measurement. Operation 1593 describes signaling at least an anticoagulant of the one or more clot-reducing agents in response to an apparent flow degradation. Operation 1597 describes causing one or more dispensations in response to an apparent problem in the flow-change-indicative measurement.

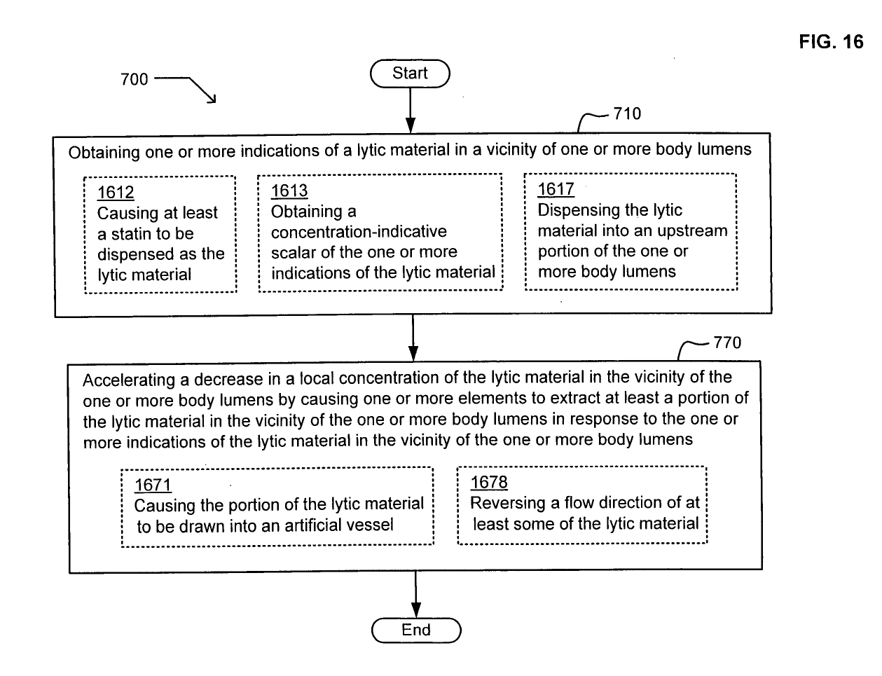

With reference now to FIG. 16, there are shown several variants of the flow 700 of FIG. 7. Operation 710—obtaining one or more indications of a lytic material in a vicinity of one or more body lumens—may (optionally) include one or more of the following operations: 1612, 1613, or 1617. In some embodiments, variants of operation 710 may (optionally) be performed by one or more instances of sensors 110, 622, response logic 155, 635, or the like as exemplified herein. Operation 770—accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens—may include one or more of the following operations: 1671 or 1678. In some embodiments, variants of operation 770 may be performed by one or more instances of extraction device 180 or the like as described herein.

Operation 1612 describes causing at least a statin to be dispensed as the lytic material (e.g. dispensing logic 610 invoking module 611 or other circuitry for actuating statin dispenser 618 or other lytic-material-containing dispenser 619 according to one or more dosage profiles in memory 621). This can occur, for example, in embodiments in which one or more instances of modules 630 are positioned (locally) upstream from a lung or other organ 660 and in which at least a portion 661 of organ 660 has been perfused with an abnormally high concentration of lytic material (relative to a time-averaged systemic normal range, for example). Alternatively or additionally, in some variants, module 690 may be configured in a context in which one or more hemorrhage-risk determinants have been established in relation to one or more other organs in a downstream vicinity 685 of lumen 695 relative to outflow 699.

Operation 1613 describes obtaining a concentration-indicative scalar of the one or more indications of the lytic material (e.g. optical or other sensors detecting a gradational concentration measurement or other concentration-indicative value). This can occur, for example, in a context in which the lytic material includes a fluorescent or other readily detected marker material.

Operation 1617 describes dispensing the lytic material into an upstream portion of the one or more body lumens (e.g. an actuator urging tissue plasminogen activator or other lytic materials locally into a carotid or pulmonary artery responsive to an indication signifying sudden, substantial, apparent decrease of blood flow through that vessel). This can occur, for example, in a context in which one or more clots have blocked a majority of flow, in which one or more complementary or systemic determinants indicate an absence of substantial hemorrhaging, and in which a care provider has specified a preset, programmatic regimen by which such material(s) will be administered in these contingencies. Such complementary determinants may include a dangerously high local blood pressure or flow in complementary arteries, for example, of the pulmonary vasculature. Such systemic determinants may include substantial increases in (resting) heart rate or substantial decreases in blood pressure over a course of minutes or hours.

Operation 1671 describes causing the portion of the lytic material to be drawn into an artificial vessel (e.g. actuator 881 allowing one or more ports 882 to draw at least some of outflow 899 into one or more vessels 883 from lumen 895). This can occur, for example, in a context in which dispenser 841 has been dispensing a therapeutic material containing one or more carcinogens or other ingredients having potentially undesirable side effects in outflow 899. Alternatively or additionally, pump 886 may be used for accelerating a decrease of the local concentration of such materials within vicinity 875.

Operation 1678 describes reversing a flow direction of at least some of the lytic material (e.g. a pump reversing a material flow direction at least through a conduit). This can occur, for example, in a context in which a flow is apparently restored, such as may be manifested in a return to a normal local pressure in a formerly-blocked vessel or in a complementary vessel.

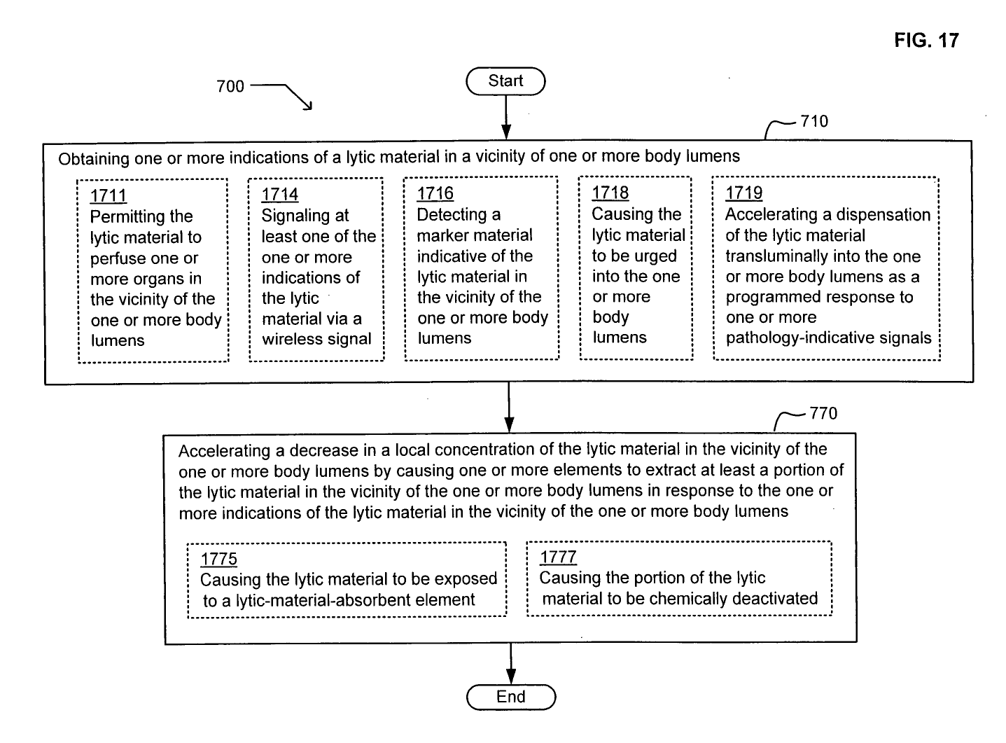

With reference now to FIG. 17, there are shown several variants of the flow 700 of FIG. 7 or 16. Operation 710—obtaining one or more indications of a lytic material in a vicinity of one or more body lumens—may (optionally) include one or more of the following operations: 1711, 1714, 1716, 1718, or 1719. In some embodiments, variants of operation 710 may (optionally) be performed by one or more instances of response logic 155, 635 or the like as exemplified herein. Operation 770—accelerating a decrease in a local concentration of the lytic material in the vicinity of the one or more body lumens by causing one or more elements to extract at least a portion of the lytic material in the vicinity of the one or more body lumens in response to the one or more indications of the lytic material in the vicinity of the one or more body lumens—may include one or more of the following operations: 1775 or 1777. In some embodiments, variants of operation 770 may be performed by one or more instances of extraction device 180 or the like as described herein.

Operation 1711 describes permitting the lytic material to perfuse one or more organs in the vicinity of the one or more body lumens (e.g. dispensing logic 610 invoking one or more dispensers 619 to inject a lytic compound or other lytic material into a renal artery or otherwise to perfuse organ 660). This can occur, for example, in an embodiment in which dispensing logic 610 can invoke other logic modules and in which system 600 implements one or more devices like those disclosed in U.S. Pat. No. 6,592,567 (“Kidney perfusion catheter”) or U.S. Pat. No. 6,514,226 (“Method and apparatus for treatment of congestive heart failure by improving perfusion of the kidney”). Alternatively or additionally, such a perfusion may reasonably be inferred at some time after a sufficiently large systemic administration of the lytic material. In some contexts this may be desirable, for example, even for a cancer patient for whom a lytic treatment in outflow 699 presents a danger. In a case in which a majority of blood flowing through module 690 is removed from a patient's vasculature into one or more conduits 667, for example, a transfusion or other blood replacement at module 690 may be provided to supplement outflow 699 (optionally with a concomitant decrease in the local concentration of the lytic material).

Operation 1714 describes signaling at least one of the one or more indications of the lytic material via a wireless signal. Operation 1716 describes detecting a marker material indicative of the lytic material in the vicinity of the one or more body lumens. Operation 1718 describes causing the lytic material to be urged into the one or more body lumens. Operation 1719 describes accelerating a dispensation of the lytic material transluminally into the one or more body lumens as a programmed response to one or more pathology-indicative signals.

Operation 1775 describes causing the lytic material to be exposed to a lytic-material-absorbent element (e.g. an actuator opening a port so that lytic-material-containing fluid comes into contact with one or more foams, fabrics, fibers, or other such fluid-absorbent materials). Operation 1777 describes causing the portion of the lytic material to be chemically deactivated (e.g. dispenser releasing protease nexin or other such plasminogen activator inhibitors). This can occur, for example, in a context in which a force apparently induced by a clot has been detectably reduced after module has dispensed a local dose of a plasminogen activator or other such lytic material. In some contexts, a quantity of the inhibitor released may be sufficient to deactivate at least 0.1% to 1% (or at most about 5% to 50%) of a released quantity of the plasminogen activator.

In light of teachings herein, and referring again to FIG. 1, those skilled in the art will recognize that any of these systems may (optionally) include a variant in receiver 146 obtains a priori implant information by receiving configuration information to describe or otherwise accommodate a lower module 190 that has been or will be implanted. This can occur, for example, in a context in which one or more instances of upper module 150 is (or will be) well situated to administer one or more lytic materials or other therapies that may be needed at one or more instances of lower module 190. Alternatively or additionally, the a priori implant information may include implant status, material reservoir status, or other such indications of modules as described herein.

Any of the above-described embodiments can likewise comprise a variant in which interface logic 140 invokes circuitry for performing operation 380 (of FIG. 3) such as one or more modules 113 of dispensing logic 115 operable for activating one or more dispensers 118, 119 when an apparent clot is detected. This can occur, for example, in a context in which the a priori implant information is embedded in circuitry or other structure of such dispensing logic 115.

Any of the above-described embodiments can likewise comprise a variant in which timing module 152 or another module 151 of response logic 155 performs operation 710 by responding to a signal from sensor 110 or some other indication that a lytic material will apparently be present in or near section 130 of lumen 195. This can occur, for example, in a context in which response logic 155 receives a notification that dispenser 119 has been activated. Alternatively or additionally, such indications may be received from one or more sensors 110 operable for detecting the lytic material directly or by detecting other such conditions as described herein. Alternatively or additionally, any of these modules or other components may likewise include a delay or other timing module 152 responsive to at least one of the one or more dispensation components. Alternatively or additionally, any of these modules or other components may likewise include one or more semi-permeable membranes 181.

Referring again to FIGS. 2-6, those skilled in the art will recognize that any of the herein-described modules or other components may likewise include one or more thrombolytic-agent-containing dispensers 228 and/or may include one or more (artificial) disposal vessels 670 and/or other features described with reference to FIG. 2 or 6. Referring again to FIG. 8, those skilled in the art will recognize that any such components may likewise include one or more disposals 888, optionally transluminal ones like disposal 889 in which one or more conduits 886 are configured to bear a blood-containing material into a body lumen. Any may likewise include one or more radiotherapy treatment modules or other such therapeutic structures 842.

Referring again to FIG. 9, alternatively or additionally, any of these modules or systems herein may likewise include an implantable, dispenser-containing valve 910. Any may likewise include one or more instances of wireless communication modules 944 for sending data to or receiving data from an outside network or other entity. Any may likewise include one or more optical sensors 975, auditory sensors 976, pressure sensors, pressure-limiting valves, strain gauges, or other such flow-force-responsive elements 978. Alternatively or additionally, any of these extraction modules or other material movement components may likewise comprise a lower-than-ambient pressure, at least initially. Alternatively or additionally, any of the above-described modules or other components may (optionally) include one or more implant-site-targeting dispensers, positioned for dispensing (a) above an implant of interest or (b) from an upstream or intermediate portion of the implant of interest.

Some or all of the embodiments described herein may generally comprise technologies for handling one or more bioactive agents and/or carriers in releasable module form, via a liquid-bearing conduit, in a mist or other spray form, in a pumped or other pressurized form, or otherwise according to technologies described herein. In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit. electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory) and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into image processing systems. That is, at least a portion of the devices and/or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting lenses to give desired focuses. A typical image processing system may be implemented utilizing any suitable commercially available components, such as those typically found in digital still systems and/or digital motion systems.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and nonvolatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Quest, Southwestern Bell, etc), or (g) a wired/wireless services entity such as Sprint, Cingular, Nextel, etc.), etc.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the ail will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A therapeutic administration system comprising:
a processor;
one or more instructions operable by the processor to obtain a priori implant information; and
one or more instructions operable by the processor to signal a decision whether to initiate implant-site-targeting treatment partly based on the a priori implant information and partly based on one or more other clot-indicative determinants.

2. The therapeutic administration system of claim 1 in which the one or more instructions operable by the processor to obtain a priori implant information comprise:
one or more instructions operable by the processor to obtain the a priori implant information from one or more implantable devices.

3. The therapeutic administration system of claim 1 in which the one or more instructions operable by the processor to obtain a priori implant information comprise:
one or more instructions operable by the processor to obtain the a priori implant information from one or more objects borne by a subject.

4. The therapeutic administration system of claim 1 in which the one or more instructions operable by the processor to obtain a priori implant information comprise:
one or more instructions operable by the processor to obtain the a priori implant information ex situ.

5. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to obtain one or more of a blood pressure indicator or a flow rate indicator of the one or more other clot-indicative determinants.

6. The therapeutic administration system of claim 1 in which the one or more instructions operable by the processor to signal a decision whether to initiate implant-site-targeting treatment partly based on the a priori implant information and partly based on one or more other clot-indicative determinants comprise:
circuitry for generating the decision whether to initiate the implant-site-targeting treatment partly in response to an implant type.

7. The therapeutic administration system of claim 1 in which the one or more instructions operable by the processor to signal a decision whether to initiate implant-site-targeting treatment partly based on the a priori implant information and partly based on one or more other clot-indicative determinants comprise:
circuitry for deciding whether to transmit one or more other treatment indications partly based on one or more hemorrhagic-stroke-indicative determinants.

8. The therapeutic administration system of claim 1 in which the one or more instructions operable by the processor to signal a decision whether to initiate implant-site-targeting treatment partly based on the a priori implant information and partly based on one or more other clot-indicative determinants comprise:
circuitry for generating the decision whether to initiate the implant-site-targeting treatment partly in response to detecting one or more emboli.

9. The therapeutic administration system of claim 1 in which the one or more instructions operable by the processor to obtain a priori implant information comprise:
one or more instructions operable by the processor to obtain an update for the a priori implant information.

10. The therapeutic administration system of claim 1 in which the one or more instructions operable by the processor to obtain a priori implant information comprise:
one or more instructions operable by the processor to obtain timing information in the a priori implant information.

11. The therapeutic administration system of claim 1 in which the one or more instructions operable by the processor to obtain a priori implant information comprise:
one or more instructions operable by the processor to obtain an implant type of the a priori implant information.

12. The therapeutic administration system of claim 1 in which the one or more instructions operable by the processor to signal a decision whether to initiate implant-site-targeting treatment partly based on the a priori implant information and partly based on one or more other clot-indicative determinants comprise:
circuitry for generating the decision whether to initiate the implant-site-targeting treatment partly in response to an apparent change in a chemical composition.

13. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to signal a decision whether to dispense one or more therapeutic materials from an implant.

14. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to signal a decision whether to dispense one or more of a thrombolytic agent or an anticoagulant.

15. The therapeutic administration system of claim 1 in which the one or more instructions operable by the processor to signal a decision whether to initiate implant-site-targeting treatment partly based on the a priori implant information and partly based on one or more other clot-indicative determinants comprise:
circuitry for generating the decision whether to initiate the implant-site-targeting treatment partly in response to an apparent change in vascular flow.

16. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to obtain one or more ischemia indicators of the one or more other clot-indicative determinants.

17. The therapeutic administration system of claim 1 in which the one or more instructions operable by the processor to signal a decision whether to initiate implant-site-targeting treatment partly based on the a priori implant information and partly based on one or more other clot-indicative determinants comprise:
a flow-force-responsive element.

18. The therapeutic administration system of claim 1 in which the one or more instructions operable by the processor to signal a decision whether to initiate implant-site-targeting treatment partly based on the a priori implant information and partly based on one or more other clot-indicative determinants comprise:
an optical sensor.

19. The therapeutic administration system of claim 1 in which the one or more instructions operable by the processor to signal a decision whether to initiate implant-site-targeting treatment partly based on the a priori implant information and partly based on one or more other clot-indicative determinants comprise:
an auditory sensor.

20. The therapeutic administration system of claim 1, further comprising:
a wireless-communication device.

21. The therapeutic administration system of claim 1, further comprising:
an implant-site-targeting dispenser responsive to the decision whether to initiate the implant-site-targeting treatment.

22-41. (canceled)

42. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to signal a decision whether to dispense one or more therapeutic materials from an implant;
one or more instructions operable by the processor to signal a decision whether to dispense one or more of a thrombolytic agent or an anticoagulant;
one or more instructions operable by the processor to obtain one or more isehemia indicators of the one or more other clot-indicative determinants; and
one or more instructions operable by the processor to obtain one or more of a blood pressure indicator or a flow rate indicator of the one or more other clot- indicative determinants.

43. A therapeutic administration system comprising:
means for obtaining a priori implant information; and
means for signaling a decision whether to initiate implant-site-targeting treatment partly based on the a priori implant information and partly based on one or more other clot-indicative determinants.

44-84. (canceled)

85. A therapeutic administration method comprising:
obtaining a priori implant information; and
signaling a decision whether to initiate implant-site-targeting treatment partly based on the a priori implant information and partly based on one or more other clot-indicative determinants.

86-115. (canceled)

116. The therapeutic administration method of claim 85 in which the obtaining a priori implant information comprises:
obtaining the a priori implant information from one or more implantable devices;
obtaining the a priori implant information ex situ;
obtaining an update for the a priori implant information;
obtaining timing information in the a priori implant information; and
obtaining an implant type.

117. The therapeutic administration method of claim 116 in which the signaling a decision whether to initiate implant-site-targeting treatment partly based on the a priori implant information and partly based on one or more other clot-indicative determinants comprises:
generating the decision whether to initiate the implant-site-targeting treatment partly in response to one or more of an apparent change in a chemical composition, an apparent change in vascular flow, the implant type, or detecting one or more emboli;
signaling a decision whether to dispense one or more therapeutic materials from an implant;
signaling a decision whether to dispense one or more of a thrombolytic agent or an anticoagulant;
obtaining one or more ischemia indicators of the one or more other clot-indicative determinants;
obtaining one or more of a blood pressure indicator or a flow rate indicator of the one or more other clot-indicative determinants; and
deciding whether to transmit one or more other treatment indications partly based on one or more hemorrhagic-stroke-indicative determinants.

118. The therapeutic administration method of claim 85 in which the signaling a decision whether to initiate implant-site-targeting treatment partly based on the a priori implant information and partly based on one or more other clot-indicative determinants comprises:
generating the decision whether to initiate the implant-site-targeting treatment partly in response to one or more of an apparent change in a chemical composition, an apparent change in vascular flow, an implant type, or detecting one or more emboli;
signaling a decision whether to dispense one or more therapeutic materials from an implant;
signaling a decision whether to dispense one or more of a thrombolytic agent or an anticoagulant;
obtaining one or more ischemia indicators of the one or more other clot-indicative determinants;
obtaining one or more of a blood pressure indicator or a flow rate indicator of the one or more other clot-indicative determinants; and
deciding whether to transmit one or more other treatment indications partly based on one or more hemorrhagic-stroke-indicative determinants.

119-322. (canceled)

323. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to obtain at least a flow rate indicator of the one or more other clot-indicative determinants.

324. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to obtain one or more ischemia indicators of the one or more other clot-indicative determinants; and
one or more instructions operable by the processor to obtain one or more of a blood pressure indicator or a flow rate indicator of the one or more other clot-indicative determinants.

325. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to signal a decision whether to dispense one or more therapeutic materials from an implant;
one or more instructions operable by the processor to obtain one or more ischemia indicators of the one or more other clot-indicative determinants; and
one or more instructions operable by the processor to obtain one or more of a blood pressure indicator or a flow rate indicator of the one or more other clot-indicative determinants.

326. The therapeutic administration system of claim 1 in which the one or more instructions operable by the processor to signal a decision whether to initiate implant-site-targeting treatment partly based on the a priori implant information and partly based on one or more other clot-indicative determinants comprise:
one or more instructions operable by the processor to generate the decision whether to initiate the implant-site-targeting treatment partly in response to an implant type and partly in response to an apparent change in vascular flow.
